(19) United States
(12) Patent Application Publication
(10) Pub. No.: US 2018/0303537 A1
(43) Pub. Date: Oct. 25, 2018

(54) Low Eletromagnetic Field Electrosurgical Cable

(71) Applicant: U.S. Patent Innovations LLC, Takoma Park, MD (US)

(72) Inventors: Jerome Canady, Lakeland, FL (US); Alexey Shashurin, West Lafayette, IN (US); Michael Keidar, Baltimore, MD (US); Taisen Zhuang, Vienna, VA (US)

(21) Appl. No.: 15/767,740

(22) PCT Filed: Oct. 17, 2016
(86) PCT No.: PCT/US2016/057310

§ 371 Date: Apr. 12, 2018

Related U.S. Application Data
(60) Provisional application No. 62/242,579, filed on Oct. 16, 2015.

(51) Int. Cl. A61B 18/04; H01B 11/18

(52) U.S. Cl. CPC A61B 18/042; A61B 2018/00166; H01B 11/1834

(57) ABSTRACT

An electrosurgical cable that produces no electromagnetic (EM) field around its vicinity (zero-EM pollution). The cable is comprised of inner insulator with embedded conductor placed inside the outer insulator tube with embedded second conductor. Sizes and materials of conductors and insulators are chosen so that voltage applied to inner conductor is higher than the breakdown voltage while voltage applied to gas gap inside the electrosurgical cable is below than the breakdown voltage. Therefore, the cable is producing discharge at the surgical handpiece, but breakdown inside the cable is prohibited.

### CROSS-REFERENCE TO RELATED APPLICATIONS

The present application claims the benefit of the filing date of U.S. Provisional Patent Application Ser. No. 62/242,579 filed by the present inventors on Oct. 16, 2016.

The aforementioned provisional patent application is hereby incorporated by reference in its entirety.

### STATEMENT REGARDING FEDERALLY SPONSORED RESEARCH OR DEVELOPMENT

None.

### BACKGROUND OF THE INVENTION

### Field of the Invention

The present invention relates to an electrosurgical cable which is not producing electromagnetic EM-field in its vicinity (zero-EM pollution) and reduces risk of electric shock for the human subjects involved in the electrosurgical procedure.

### Background of the Related Art

Electrosurgical cables are used to connect an electrosurgical generator to an electrosurgical surgical hand piece and deliver high voltage and gas flow from the generator to the electrosurgical handpiece. Conventional electrosurgical cables utilized in electrosurgical systems consist of one high voltage electrode placed inside an electrically insulating flexible tube. The high voltage electrode inside the insulting tube creates strong electromagnetic (EM) field around the cable. Frequencies of the electrosurgical generators are below 1 MHz, which is associated with wavelengths λ>300 m. Therefore, a conventional one-electrode electrosurgical cable effectively is a short antenna with length L<<λ. Radiated EM power is low since antenna in far from the resonance; however, values of the electric field in the near-zone of the antenna are high due to high voltages applied to the electrode. Local electric fields can be as high as E˜V/D˜1000 V/cm, taking very realistic separation between the electrosurgical cable and patient D˜1 cm, that can readily appear during the electrosurgical procedure when cable is constantly moving with respect to the patient.

### SUMMARY OF THE INVENTION

The present invention relates to an electrosurgical cable that connects between an electrosurgical unit and a handpiece or housing that does not produce an EM-field or only a negligible EM-field in its vicinity. The cable can be used with any electrosurgical generator. The cable is intended to simultaneously deliver gas flow and high voltage electrical energy required for electrosurgical unit operation. Conventional electrosurgical cables utilized in the electrosurgical probes use only one conductor inside the insulating tube to which high voltage is applied. In contrast, present invention utilizes two conductors, namely an inner high voltage conductor and an outer conductor (connected to patient pad). The critical feature of present invention is that inner conductor electrical insulation that provides the following critical function. High voltage applied to the central electrode (U₀) is chosen above the breakdown threshold (U_{BD}) in order to initiate discharge on the electrosurgical handpiece (U₀>U_{BD}). However, inside the cable a significant fraction of the applied voltage drops on the inner insulator, so that remaining voltage applied to the gas gap (U_{gas}) is below the breakdown threshold: U_{gas}<U_{BD}. This allows prevention of breakdown and ignition of plasma discharge inside the cable.

The present invention has two important benefits in comparison with conventional electrosurgical cables. First, the present cable is completely shielded and therefore it does not produce EM-field around itself in contrast with conventional electrosurgical probe cables which produce EM-field as regular short dipole antenna. Second, the present electrosurgical cable significantly reduces risk of electric shock of human subjects involved in electrosurgical procedure. Indeed, conventional electrosurgical cables can possess significant risk of electrical shock in case outer insulation layer is compromised. In contrast, compromising any insulators in the case when present invention is used may either cause human contact with shielded electrode or create short-circuit of the electrosurgical unit. Both events are electrically safe for the involved human subjects.

In a preferred embodiment the present invention is an electrosurgical cable having an elongated outer conductor, an outer insulator surrounding said outer conductor, said outer conductor and said outer insulator forming a tube, an elongated inner conductor inside said tube, and an inner insulator surrounding said inner conductor. There is a channel between and interior surface of said tube and said inner insulator. Further, sizes and materials of conductors and insulators are chosen so a voltage applied to the inner conductor is higher than the breakdown voltage and a voltage applied to gas flowing within said channel is below than the breakdown voltage. The electrosurgical cable may further have an electrical connector connected to said inner electrode for connecting said inner electrode to an electrosurgical power supply, an electrical connector connected to said out electrode for connecting said outer electrode to a ground, and a fluid connector connected to aid tube for connecting said tube to a fluid source.

In another preferred embodiment, the present invention is an electrosurgical cable having an elongated outer conductor having an outer radius c, an outer insulator surrounding said outer conductor and having inner radius d and an outer radius e, said outer conductor and said outer insulator forming a tube, an elongated inner conductor inside said tube, said inner electrode having a radius a, an inner insulator surrounding said inner conductor, said inner insulator having an outer radius b. There is a channel between and interior surface of said tube and said inner insulator and the radii a, b, c, d, e are selected so a<b<c≤d≤e. Further, the radii a, b, c, d and e are selected so a total applied voltage (U₀) is distributed between the inner insulator (Uᵢₙ) and gas gap between inner and outer insulators (U_{gas}), so that U₀=Uᵢₙ+U_{gas}. Still further, a, b, c, d and e may be selected so that Uᵢₙ×U_{gas}. For example, the radii may be selected as follows: a=0.25 mm, b=2.5 mm, c=d=4 mm and e=5 mm.

### BRIEF DESCRIPTION OF THE FIGURES

For a more complete understanding of the present invention and the advantages thereof, reference is now made to the following description and the accompanying drawings, in which:

FIG. 1 is a perspective view of an electrosurgical cable in accordance with a preferred embodiment of the present invention with connectors on one end of the cable and an electrosurgical handpiece on the other end of the cable.

FIG. 2 is a perspective view of a portion of a cable in accordance with a preferred embodiment of the present invention.

FIG. 3 is a cross-section of a cable in accordance with a preferred embodiment of the present invention showing relationships of dimensions and voltage drops of various component parts of the cable.

### DETAILED DESCRIPTION OF THE PREFERRED EMBODIMENT

In describing a preferred embodiment of the invention illustrated in the drawings specific terminology will be resorted to for the sake of clarity. However, the invention is not intended to be limited to the specific terms so selected, and it is to be understood that each specific term includes all technical equivalents that operate in similar manner to accomplish a similar purpose. The preferred embodiment of the invention is described for illustrative purposes, it being understood that the invention may be embodied in other forms not specifically shown in the drawings.

The present invention presents a novel concept of an electrosurgical cable which produces no EM-field or only negligible EM-field around itself (zero-EM pollution) and offers operation without risk of electric shock for human subjects involved in the electrosurgical procedure.

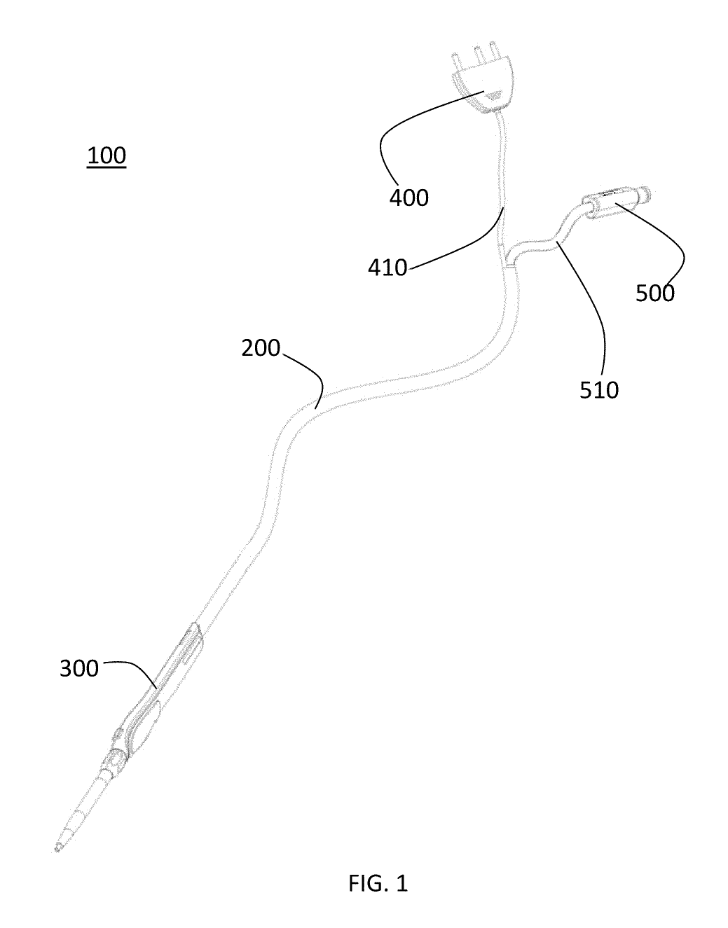

As shown in FIG. 1, a cable in accordance with the present invention can be used in an electrosurgical system, which, for example, may be a cold plasma electrosurgical system, a hybrid plasma electrosurgical system, or an argon coagulation electrosurgical system. The cable 200 of the present invention may have an electrical connector 400 and a gas supply connector 500 on one end and a handpiece 300 on its other end. The electrical connector will have wiring 410 from the cable 200 and the gas connector will have a tube 510 from the cable 200. Various known connectors 400 and 500 may be used with the present invention.

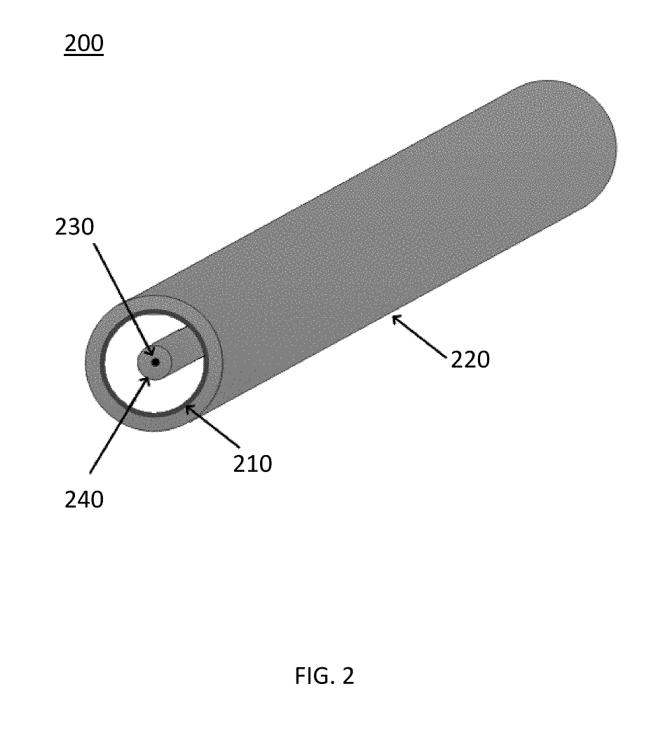

As shown in FIG. 2, the cable 200 has an inner electrode 230 to be connected to an electrosurgical generator, surrounded by insulation 240. When the cable is in use, this inner electrode 230 would be connected to a power supply through connector 400. In the preferred embodiment, the electrode 230 is made of cylindrical stainless steel wires of 0.25 mm radius embedded in silicon rubber insulator with radius about 2.5 mm. Material and diameter of the wire is not limited to utilization of stainless steel and other electrically conducting materials can be used as well. Preferentially, diameter (a) of the wire 230 should be chosen depending on precise maximal current requirements of the specific electrosurgical system. Radius (b) of insulator 240 and its material can be varied in wide range as well. In the preferred embodiment silicon rubber was used as material for insulator 240 having relative dielectric permittivity ε˜3, however dielectrics with other values of can be utilized as well. Preferentially, flexible electrically insulating material should be used to provide electrical insulation along with good flexibility of the electrosurgical cable as a whole.

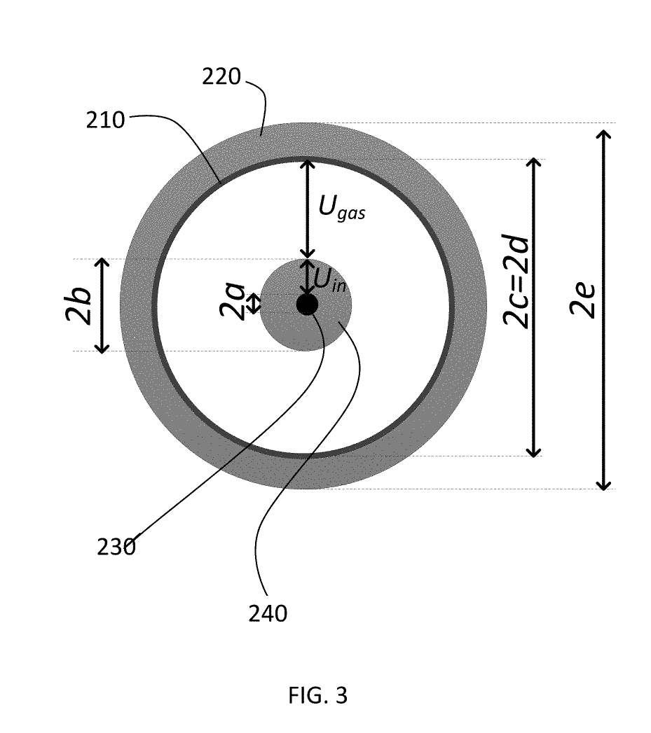

The cable further has an outer electrode 210 to be connected to a ground, surrounded on its exterior by electrical insulator 220. As shown in FIGS. 2-3, the outer conductor is cylindrical and forms a tube within which the inner conductor and inner insulation are placed such that a fluid channel is formed between the outer conductor 210 and the inner insulator 240. In the preferred embodiment, the outer electrode 210 is made of stainless steel braided sleeving embedded into outer insulating tube 220. Transparency of the braided shield can be varied depending on requirements of maximal cable weight. Lighter electrosurgical cables can be obtained by reducing diameter of the wire used in the braid and increasing of its transparency. Thin foil or other form of outer conductor can be used as well. Minimal cross-section of the outer conductor 210 should be limited by maximal electric current values required to be drawn through the particular electrosurgical cable. Inner radius (c) of the outer conductor 210 and outer radiuses (e) of insulator 220 and its material can be varied. Preferentially, flexible electrically insulating material should be used to provide electrical insulation along with good flexibility of the electrosurgical cable. The braided shield can be embedded inside the outer conductor and can have radius (d) in the range c≤d≤e. Note, FIG. 3 shows the case when inner diameter of the outer conductor 210 is shown to be exactly equal to diameter of outer conductor 220 (c=d) and inner electrode 230 and the outer tube are coaxial.

In a preferred embodiment, the inner conductor and outer conductor are cylindrical but other shapes may be sued with the invention.

In preferred embodiment Helium was used as working gas while other gases such as Argon can be used as well.

Relative sizes of the conductors 210, 230 and insulators 220, 240 should be chosen so that a<b<c≤d≤e. In preferred embodiment it was chosen a=−0.25 mm, b=2.5 mm, c=d=4 mm and e=5 mm.

The total applied voltage (U₀) is distributed between the inner insulator (Uᵢₙ) and gas gap between inner and outer insulators (U_{gas}), so that U₀=Uᵢₙ+U_{gas} as shown in FIG. 2. Ratio of voltages Uᵢₙ and U_{gas}, can be expressed as:

$$\frac{{U}_{in}}{{U}_{gas}} = \frac{ln \frac{b}{a}}{ɛln \frac{c}{b}}$$

In preferred embodiment, the ratio

$$\frac{{U}_{in}}{{U}_{gas}} = 1.1$$

meaning that Uᵢₙ≈U_{gas} and thus using U₀=Uᵢₙ+U_{gas} one can obtain the that

$${U}_{in} \approx {U}_{gas} \approx \frac{{U}_{0}}{2} .$$

In preferred embodiment, U₀≤4 kV was used and U_{BD} was about 2.5 kV. Therefore, Uᵢₙ×U_{gas}≤2 kV and thus U_{gas}<U_{BD} providing that breakdown inside the electrosurgical cable prohibited. At the same time, U₀>U_{BD} and thus the voltage is sufficient to produce breakdown at the surgical handpiece. Various combinations of radiuses and dielectric permittivity can be used, however, it is critical to choose theses parameters so that two conditions are simultaneously satisfied:
1. U₀>U_{BD}—voltage is sufficient to produce breakdown at the surgical handpiece
2. U_{gas}<U_{BD}—breakdown inside the electrosurgical cable is prohibited

In the preferred embodiment, the inner electrode with insulator was freely placed inside the outer tube. However, relative location of the inner electrode with insulator with respect to the outer tube could be different such as coaxial or any other relative positioning. Also, inner insulator can be either permanently attached or not attached to the inner wall of the outer insulator.

The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

1. An electrosurgical cable comprising:
an elongated outer conductor;
an outer insulator surrounding said outer conductor, said outer conductor and said outer insulator forming a tube;
an elongated inner conductor inside said tube;
an inner insulator surrounding said inner conductor, wherein there is a channel between an interior surface of said tube and said inner insulator
an electrical connector connected to said inner conductor for connecting said inner conductor to an electrosurgical power supply;
an electrical connector connected to said outer conductor for connecting said outer conductor to a ground; and
a fluid connector connected to said tube for connecting said tube to a fluid source.

2. An electrosurgical cable according to claim 1 wherein sizes and materials of conductors and insulators are chosen so a voltage applied to the inner conductor is higher than the breakdown voltage and a voltage applied to gas flowing within said channel is below than the breakdown voltage.

3. (canceled)

4. An electrosurgical cable comprising:
an elongated outer conductor having an outer radius c;
an outer insulator surrounding said outer conductor and having inner radius d and an outer radius e, said outer conductor and said outer insulator forming a tube;
an elongated inner conductor inside said tube, said inner electrode having a radius a; and
an inner insulator surrounding said inner conductor, said inner insulator having an outer radius b;
wherein there is a channel between an interior surface of said tube and said inner insulator and the radii a, b, c, d, e are selected so a<b<c≤d≤e and wherein the radii a, b, c, d and 3 are selected so a total applied voltage (U₀) is distributed between the inner insulator (Uᵢₙ) and gas gap between inner and outer insulators (U_{gas}), so that U₀=Uᵢₙ+U_{gas}.

5. (canceled)

6. An electrosurgical cable according to claim 4 wherein a, b, c, d and e are selected so that Uᵢₙ≈U_{gas}.

7. An electrosurgical cable according to claim 5 wherein a=0.25 mm, b=2.5 mm, c=d=4 mm and e=5 mm.

8. An electrosurgical cable comprising:
an elongated outer conductor having an outer radius c;
an outer insulator surrounding said outer conductor and having inner radius d and an outer radius e, said outer conductor and said outer insulator forming a tube;
an elongated inner conductor inside said tube, said inner electrode having a radius a; and
an inner insulator surrounding said inner conductor, said inner insulator having an outer radius b;
wherein there is a channel between and interior surface of said tube and said inner insulator and the radii a, b, c, d, e are selected so a<b<c<d≤e and a=0.25 mm, b=2.5 mm, c=d=4 mm and e=5 mm.

9. An electrosurgical cable according to claim 8 further comprising:
an electrical connector connected to said inner conductor for connecting said inner conductor to an electrosurgical power supply;
an electrical connector connected to said outer conductor for connecting said outer conductor to a ground; and
a fluid connector connected to said tube for connecting said tube to a fluid source.

10. An electrosurgical cable according to claim 4 further comprising:
an electrical connector connected to said inner conductor for connecting said inner conductor to an electrosurgical power supply;
an electrical connector connected to said outer conductor for connecting said outer conductor to a ground; and
a fluid connector connected to said tube for connecting said tube to a fluid source.
